(19) United States
(12) Patent Application Publication
(10) Pub. No.: US 2002/0099277 A1
(43) Pub. Date: Jul. 25, 2002

(54) Disposable vital signs monitoring sensor band with removable alignment sheet

(75) Inventors: Andrea Harry, Cambridge (GB); Michael Sheldon, Cambridge (GB); Graham Lay, Cambs (GB); Harpal Kumar, Cambridge (GB)

Correspondence Address: Woodcock Washburn Kurtz, Mackiewicz & Norris LLP, One Liberty Place - 46th Floor, Philadelphia, PA, 19103, US

(73) Assignee: Nexan Limited

(21) Appl. No.: 09/950,961

(22) Filed: Sep. 12, 2001

Related U.S. Application Data
(63) Continuation-in-part of application No. 09/659,301, filed on Sep. 12, 2000.

(51) Int. Cl. A61B 5/00

(52) U.S. Cl. 600/301

(57) ABSTRACT

A cordless, disposable sensor band containing sensors for acquiring physiological parameters from a patient characterized by an alignment mechanism that assures proper alignment of the sensor band when the sensor band is applied by medical or nonmedical personnel. A release liner is applied to a side of the sensor band away from the skin. The release liner has alignment features that provide for easy horizontal and vertical alignment of the release liner during application. Once the sensor band and release liner are in place and attached to the patient's skin, the release liner is peeled away leaving the sensor band for collecting physiological parameter data. Breathing may be monitored by measuring the impedance diagonally across the patient's chest from a sensor on one breast to a sensor placed under the opposite axilla using the alignment features on the release liner.

### CROSS REFERENCE TO RELATED APPLICATIONS

The present patent application is a continuation-in-part of U.S. patent application Ser. No. 09/659,301, filed Sep. 12, 2000, and entitled “Disposable Vital Signs Monitoring Sensor Band with Removable Alignment Sheet” the contents of which are hereby incorporated by reference in their entirety.

### BACKGROUND OF THE INVENTION

1. Field of the Invention

The present invention relates to a disposable vital signs monitoring sensor band for use in a system that monitors vital signs and captures data from a patient. In particular, the present invention is a disposable vital signs monitoring sensor band with a removable alignment release liner that enables patients to properly apply, without the assistance of medical personnel, the sensor band to the patient's body for the remote electronic capture of noninvasive vital signs data including, e.g., full waveform ECG, respiration, blood oxygenation, and the like. Respiration is measured using a diagonal impedance technique that accounts for movement of the chest and abdomen with a single set of sensors.

2. Description of the Prior Art

Remote patient monitoring techniques are generally known in which electrodes are placed on the patient to monitor the patient's vital signs and the captured data is transmitted to a remote display for monitoring the patient's condition. Remote monitoring systems are known which permit a doctor or nurse to monitor the conditions of several hospitalized patients from a central monitoring site in the hospital. Typically, sophisticated patient monitoring equipment is used to collect data from the patient, and the collected data is transmitted via wire to the central monitoring site in the hospital. Generally, wireless systems are problematic in the hospital setting because of the proximity of the respective patients and the amount of interference found in such a setting.

Remote monitoring of patients' vital signs from their homes introduces an entirely new set of challenges for transmitting the gathered data to a central location for evaluation. Numerous attempts have been made to facilitate such data collection and transmission; however, in each case, cumbersome and uncomfortable monitoring equipment is placed on the patient and the patient is tethered to the monitoring equipment by electrical cords, thereby limiting the patient's movement. In some prior art systems, the electrical cords have been removed and the transmissions to the monitoring equipment made using telemetry techniques. Unfortunately, such systems limit the movement of the patient to a limited area near the vital signs monitor.

On the other hand, Bornn et al. describe a portable physiological data monitoring/alert system in U.S. Pat. Nos. 4,784,162; 4,827,943; 5,214,939; 5,348,008; 5,353,793; and 5,564,429 in which one or more patients wear sensor harnesses including a microprocessor which detects potentially life-threatening events and automatically calls a central base station via radiotelemetry using a radio modem link. In a home or alternate site configuration, communications between the base station and remote unit is by way of commercial telephone lines. Generally, the system automatically calls “911” or a similar emergency response service when an abnormality is detected by the ECG monitor. Unfortunately, the sensor harness is quite cumbersome and conspicuous and uncomfortable for the patient.

Segalowitz discloses a wireless vital signs monitoring system in U.S. Pat. Nos. 4,981,141; 5,168,874; 5,307,818; and 5,511,553 including a precordial strip patch having a multi-layer flexible structure for telemetering data by radio frequency or single wire to hardware recording apparatus and a display monitor. Microsensors and conductive contact elements (CCEs) are mounted on the strip patch so as to permit simultaneous and continuous detection, processing and transmission of 12-lead ECG, cardiac output, respiration rate, peripheral blood oximetry, temperature of the patient, and ECG fetal heart monitoring via a single wavelength of radio frequency transmission. While the precordial strip patch used by Segalowitz purportedly transmits vital signs data up to 50 meters, it requires a dual-stage operational amplifier chip, an encoder modulator chip, a wireless transmitter chip including an oscillator, and other costly components such as artificial intelligence software, sound and visual alarms, and a microprocessor. As a result, the precordial strip patch is relatively expensive to manufacture and operate. Also, as with the other telemetry systems noted above, the emphasis of Segalowitz is on real-time monitoring and alerting of medical personnel to immediate medical needs of the patient. Moreover, the precordial strip patch generally must be applied by a knowledgeable physician or nurse since the average patient would not know how to properly place the precordial strip patch for collection of the vital signs data and the precordial strip patch has no alignment features.

Platt et al. also disclose a sensor patch for wireless physiological monitoring of patients in U.S. Pat. No. 5,634,468. Platt et al. describe a sensor and system for monitoring ECG signals remotely from patients located in non-hospital sites. In this system, a sensor patch containing sensing electrodes, signal processing circuitry and radio or infra-red transmission circuitry is attached to the patient's body and preferably worn for at least a week before its power supply is exhausted and the sensor patch is thrown away. A receiver at a primary site in the vicinity of the patient receives the data transmitted by the sensor patch and stores the sensed data. When the patient feels discomfort or concern, or if the portable unit sounds an alarm, the patient telephones the monitoring station and downloads the stored data from the portable unit via the standard voice telecommunications network. The downloaded ECG data is then monitored and analyzed at the monitoring station. The receiver in the proximity of the patient may be a portable unit carried around by the patient, where the portable unit includes a receiver, a processor for processing the received data to identify abnormalities, a memory for storing the sensed data, and circuitry for interfacing to a telephone line to send the ECG data signals to the monitoring station. The monitoring station decodes the received ECG signals and performs beat and rhythm analysis for classification of the ECG data. If an abnormal condition is discovered, medical personnel in the vicinity of the patient are contacted. As with the Segalowitz precordial strip patch, the sensor patch generally must be carefully placed by trained medical personnel.

In commonly owned U.S. patent application Ser. Nos. 09/292,405, 09/292,159, 09/292,157, and 09/591,597, the contents of which are hereby incorporated by reference in their entireties, a remote telemonitoring system and associated disposable sensor band are described for use to continuously collect vital signs data from a patient without interaction with medical personnel. Since the disposable sensor band is generally applied by the patient without interaction with medical personnel, it has been found that the disposable sensor band may not always be applied properly by the patient for data collection. Moreover, since not all patients are of the same sizes and shapes, application of the disposable sensor band proved to be difficult for certain patients. An improved method for applying such disposable sensor bands is desired that makes the placement of the disposable sensor bands intuitive and simple for the patients. The present invention has been designed to address this need in the art.

### SUMMARY OF THE INVENTION

The present invention meets the above-mentioned need in the prior art by providing an improved application technique and associated apparatus whereby any patient may easily apply such disposable sensor bands with proper alignment for vital signs monitoring.

The invention is an adhesive, preferably cordless, disposable sensor band with electrode patches, other sensors, and a connector dock for accepting a electronics unit referred to herein as an “epatch” that collects, stores, and/or transmits detected vital signs data. The sensor band is easy-to-use and is positioned on the patient by the patient. The sensor band is designed to be worn comfortably by the patient for 24 hours, at which time the sensor band may be discarded and replaced by a new sensor band. The epatch is ideally designed to store and/or transmit all vital signs data generated by the patient during that 24-hour period to a personal data logger located on or near the patient. The epatch is removed from the sensor band before the sensor band is discarded, and the epatch is reused. Since the vital signs data is collected on the patient or near the patient, the patient is free to move around while his or her vital signs are being monitored.

In a preferred embodiment, a release liner is applied to a surface of the sensor band away from the patient's skin to facilitate application of the sensor band by the patient. Once the sensor band is properly placed on the patient's skin, the release liner is removed and discarded. The release liner is specially shaped to have registration points for use by the patient in assuring that the sensor band is properly placed on the patient's chest or other position prior to activation. For example, for a sensor band to be applied to the chest, the release liner preferably includes a shaped neck section to align with the patient's collarbones to provide vertical alignment and a mid chest pointer to provide horizontal alignment. The release liner further includes a marking on an underarm section of the release liner which, when aligned with a marker on the mid chest pointer, provides for the correct positioning of the underarm section relative to the patient's body. A neck loop may also be used to support the sensor band in place while it is being positioned on the patient's chest.

The sensor band preferably includes a breathable membrane having holes that permit alignment of the membrane and the attached electrodes to the release liner during manufacture. A polyester layer is also used between the at least one electrode and the membrane to provide rigidity to the sensor band. Similarly, holes in the membrane may be used to align the sensor band with body markings on the patient's skin so that a release liner is not necessary.

The scope of the invention also includes a method for positioning a band containing at least one physiological sensor relative to the patient's body. A method in accordance with an aspect of the invention comprises the following steps: aligning a tab portion of the release liner with the patient's sternum; aligning a first marker on an underarm section of the release liner with a second marker on the tab portion of the release liner; attaching the band to the patient's skin; and removing the release liner.

Proper placement of the electrodes using the alignment method of the invention also permits the measurement of “diagonal impedance” signals between an electrode on the patient's breast and an electrode under the patient's axilla on the opposite side of the patient's body. When measured using the sensor band of the invention, these “diagonal impedance” signals are found to correlate quite nicely with air flow measurements of air into and out of the patient's lungs, thereby permitting the measurement of breathing and the detection of apnea events.

### BRIEF DESCRIPTION OF THE DRAWINGS

The above and other objects and advantages of the invention will become more apparent and more readily appreciated from the following detailed description of a presently preferred exemplary embodiment of the invention taken in conjunction with the accompanying drawings of which:

FIG. 1 is a general block diagram of a disposable sensor band in accordance with the invention.

FIG. 2(a) illustrates the top side of the sensor band (away from the patient's skin) including electrodes for attachment to the patient's body for measuring vital signs data such as full waveform single or multiple lead ECG, full waveform respiration, and blood oxygen levels using the techniques of the invention.

FIG. 2(b) illustrates the back side of the sensor band (towards the patient's skin) of FIG. 2(a) including electrodes for attachment to the patient's body for measuring vital signs data such as full waveform single or multiple lead ECG, full waveform respiration, and blood oxygen levels using the techniques of the invention.

FIG. 3 illustrates a preferred embodiment of a release liner on which the sensor band of FIGS. 2(a) and 2(b) is placed for application to the patient's chest by the patient in accordance with the techniques of the invention.

FIGS. 4(a)-4(h) illustrate the method of constructing the disposable sensor band in accordance with the invention.

FIGS. 5(a) and 5(b) illustrate the technique for applying the disposable sensor band of the invention by aligning the release liner with the patient's collarbones and sternum and then removing the release liner.

FIG. 6 illustrates respiration traces taken from a patient using the disposable sensor band of the invention and the correlation of these respiration traces with the measured airflow data to/from the patient.

### DETAILED DESCRIPTION OF PRESENTLY PREFERRED EMBODIMENTS

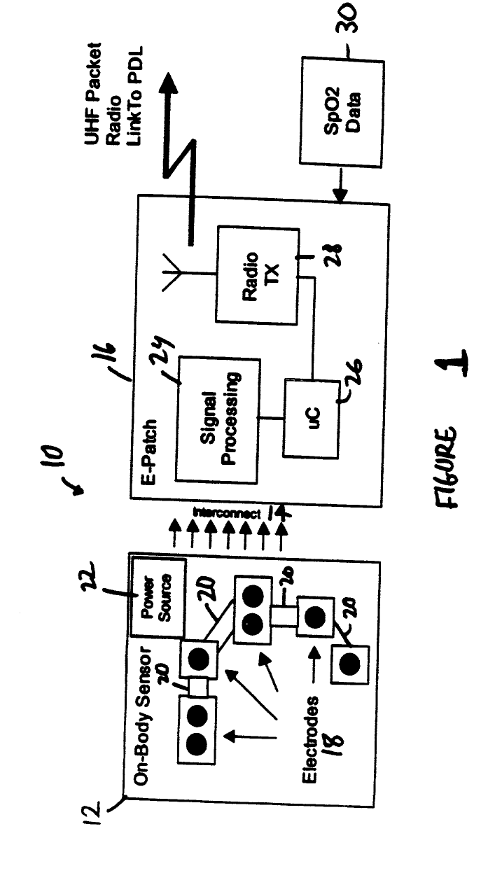

A system and method with the above-mentioned beneficial features in accordance with a presently preferred exemplary embodiment of the invention will be described below with reference to FIGS. 1-6. It will be appreciated by those of ordinary skill in the art that the description given herein with respect to those figures is for exemplary purposes only and is not intended in any way to limit the scope of the invention. All questions regarding the scope of the invention may be resolved by referring to the appended claims.

As described in detail in the aforementioned commonly owned U.S. patent application Ser. Nos. 09/292,405, 09/292,159, 09/292,157, and 09/591,597, the disposable sensor band is designed to extend across the patient's chest and includes electrodes and other sensors which are situated so as to measure full waveform single or multiple lead ECG, full waveform respiration (using impedance and/or resistance bend sensor), and perhaps other physiological parameters such as skin temperature and motion. Of course, other vital signs, such as EEG and blood oxygenation (SpO₂), could be measured as desired using sensors included within the existing sensor band and placed either on the chest or elsewhere on the body, or using sensors in another sensor band placed either on the chest or elsewhere on the patient's body. Conventional blood oxygenation sensors placed on the finger, wrist, or ear may also provide data through a wire or wireless link to the sensor band. Signal processing circuitry of the sensor band receives the sensor data and is powered by, e.g., a zinc-air battery pack in the sensor band to permit the sensor band to collect vital signs data for approximately 30 hours and to store the collected vital signs data in the epatch and/or to transmit the data to a personal data logger of the type described in U.S. patent application Ser. Nos. 09/292,158 or 09/590,996, the contents of which are hereby incorporated by reference in their entireties, that may be carried by the patient or located nearby. The sensor band is typically removed and disposed of every 24 hours and replaced by a new sensor band.

As will be explained in more detail below, the sensor band is designed such that the patient only has to prepare his or her skin, peel back a protective strip over the hydrogel and membrane adhesive layers which are, in turn, placed over the electrodes, and stick the sensor band 10 to the prepared skin in a position for measurement of the vital signs such as ECG. As also described in detail below, a release liner is placed over the sensor band during application by the patient to facilitate alignment of the sensor band on the patient's chest. Preferably, the sensor band is provided in a number of sizes sufficient to administer to infants as well as large adults.

FIG. 1 is a general block diagram of a disposable sensor band 10 in accordance with the invention. As shown, the sensor band 10 in a preferred embodiment includes a disposable on-body sensor portion 12 connected via interconnect 14 to a reusable electronics portion (“epatch”) 16. As shown, on-body sensor 12 includes electrodes 18 connected by circuit traces 20 and a power source 22 that powers the electronics of epatch 16 when the epatch is connected to the on-body sensor 12 via interconnect 14. Epatch 16 includes signal processing circuitry 24 that receives, amplifies, filters, processes the analog data from the sensors 18, performs analog to digital data conversion at 10 bit accuracy (with subsequent reduction for all but the ECG signal), and the like, and a microcontroller 26 that formats the data stream from the signal processing circuitry 24 for transmission via radio transmitter 28 to a personal data logger or other mechanism for storing the vital signs data. Of course, the functionality of the signal processing circuitry 24 and the microcontroller 26 may be performed by one or more ASICs or by one or more micro-controllers using techniques known in the art. As shown, a SpO₂ monitoring device 30 including an ear clip or finger sensor also may provide SpO₂ data to the epatch 16 for transmission using techniques known in the art. More details concerning the design and manufacture of the sensor band 10 can be found in U.S. patent application Ser. Nos. 09/292,159, 09/292,157.

The disposable sensor band 10 of the invention thus includes on-body sensor 12 and an electronics unit or “epatch” 16 that are jointly responsible for the collection of the required physiological parameters and wired or wireless broadcast of the collected data to a personal data logger or other monitoring device. In accordance with the invention, an improved application technique is employed to assure that the on-body sensor 12 is correctly positioned on the patient's body and adhered to the patient's body so that the sensor electrodes 18 are correctly positioned to collect the physiological parameters. In a preferred embodiment, the on-body sensor 12 also includes an interconnect for a flying lead electrode to enable a third view of the heart to be monitored using the disposable sensor band 10. The integrated power source 22 is preferably mounted in interconnect 14 and preferably includes sufficient capacity to permit at least 24 hours of “epatch” operation, including power provision for external SpO² monitoring device 30, if provided. Since power is only provided to the electronics when attached, disconnection of the epatch 16 provides a method of preventing continued data capture and transmission when not desired.

The epatch 16, on the other hand, processes the ECG, respiration, and SpO₂ data and encodes the data into digital form. In a preferred embodiment, an identifier is attached to the data to identify the source of the data prior to transmission of packetized data using a unidirectional UHF radio link provided by radio transmission circuitry 28. Preferably, the epatch 16 is easy to connect to the on-body sensor 12 using interconnect 14 and the epatch 16 also performs self-testing on power up and transmits the result to verify proper operation.

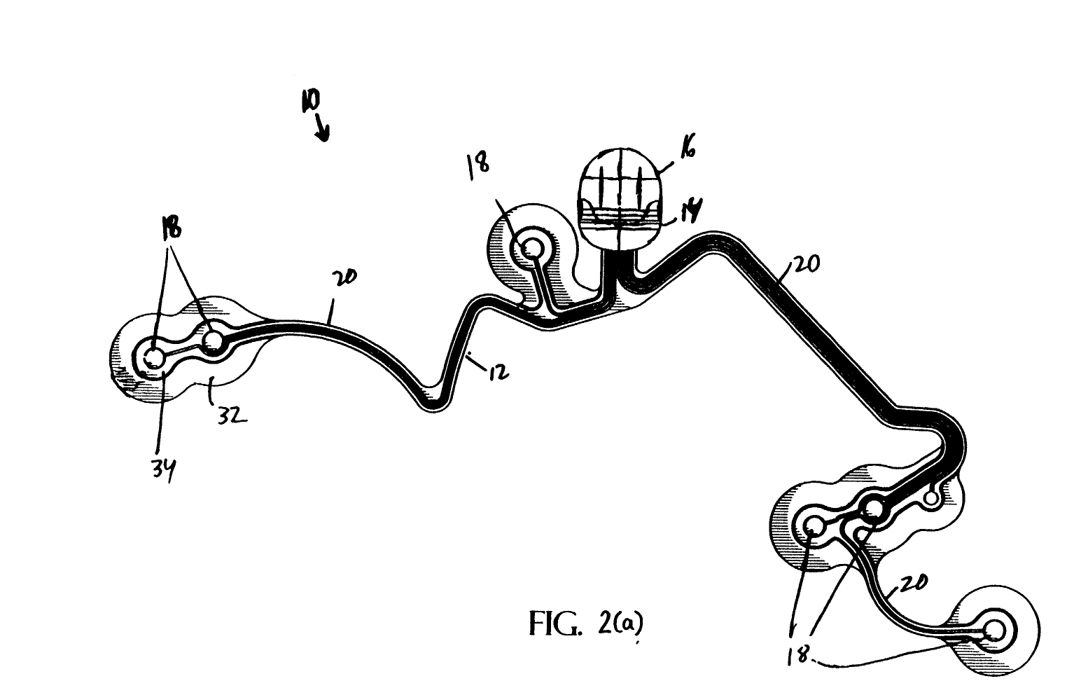

FIG. 2(a) illustrates the top side of the disposable sensor band 10 (away from the patient's skin) including electrodes 18 for attachment to the patient's body for measuring vital signs data such as full waveform single or multiple lead ECG or full waveform respiration using the techniques of the invention, while FIG. 2(b) illustrates the back side of the disposable sensor band 10 (towards the patient's skin). In accordance with the invention, the sensors 18 are placed in the appropriate precordial positions for measuring the desired physiological parameters such as ECG data. Since the spacing between the precordial positions may vary for patients depending upon size, the disposable sensor band 10 preferably comes in a range of sizes to accommodate patients of different sizes.

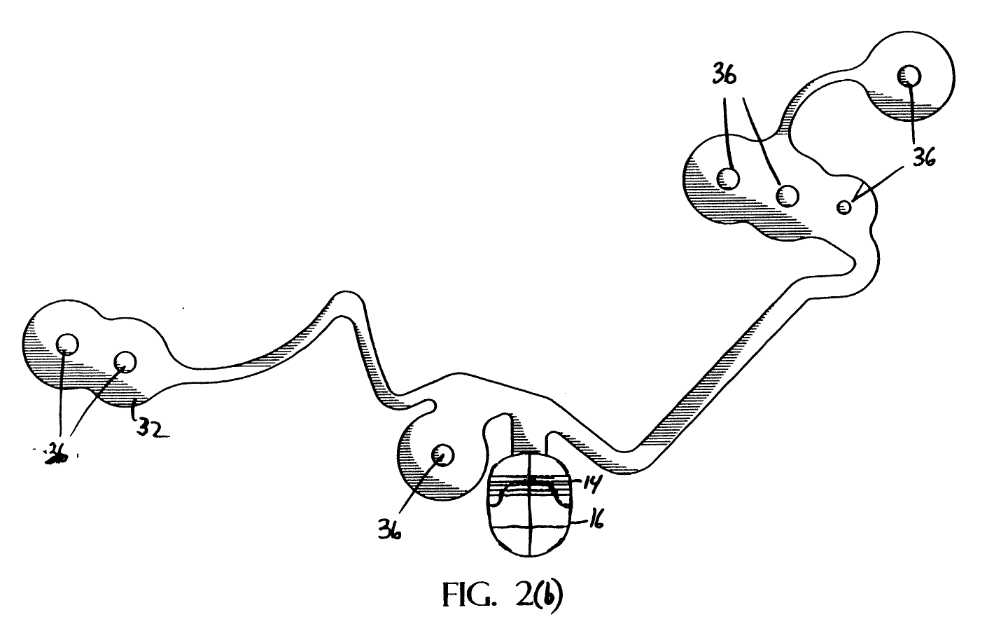

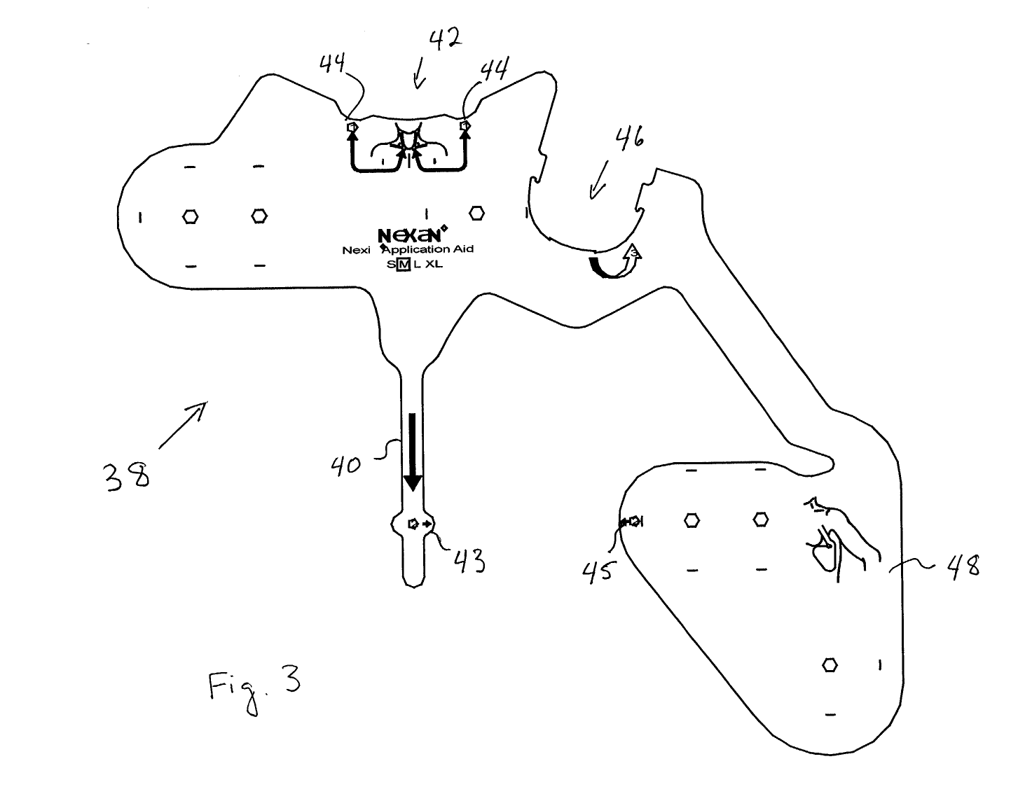

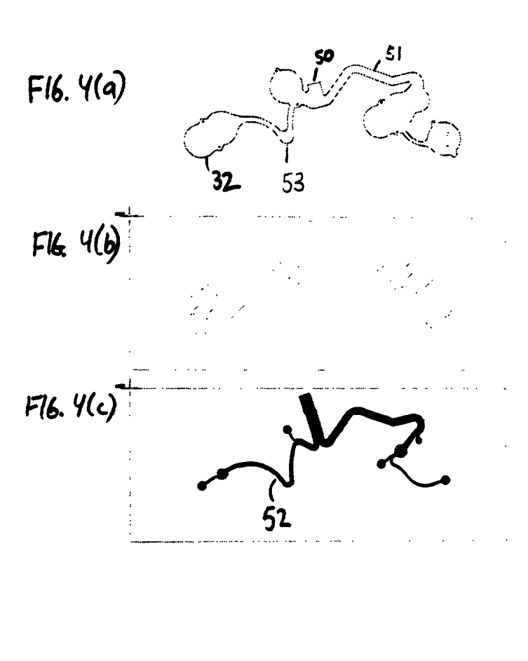

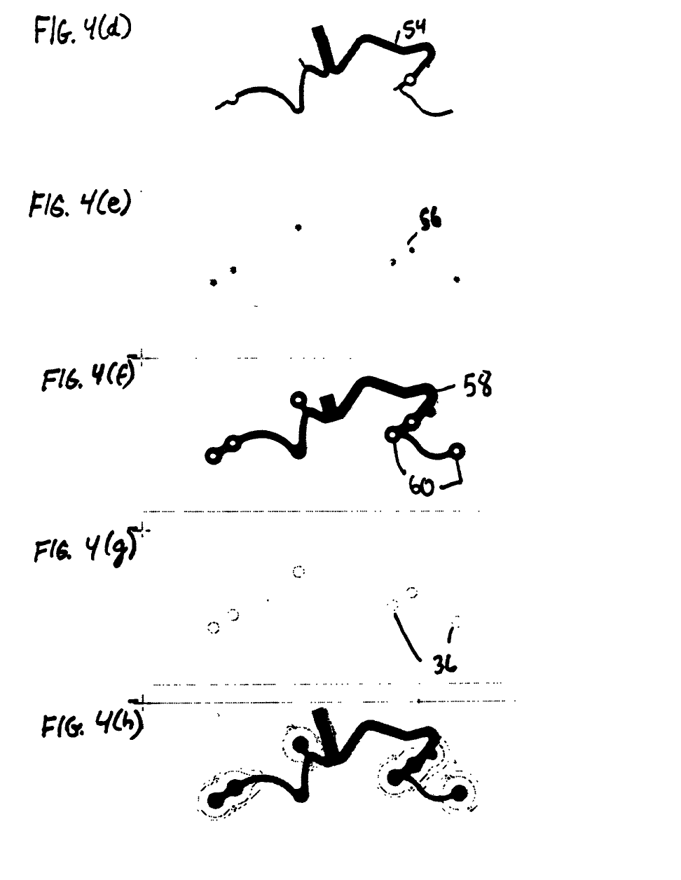

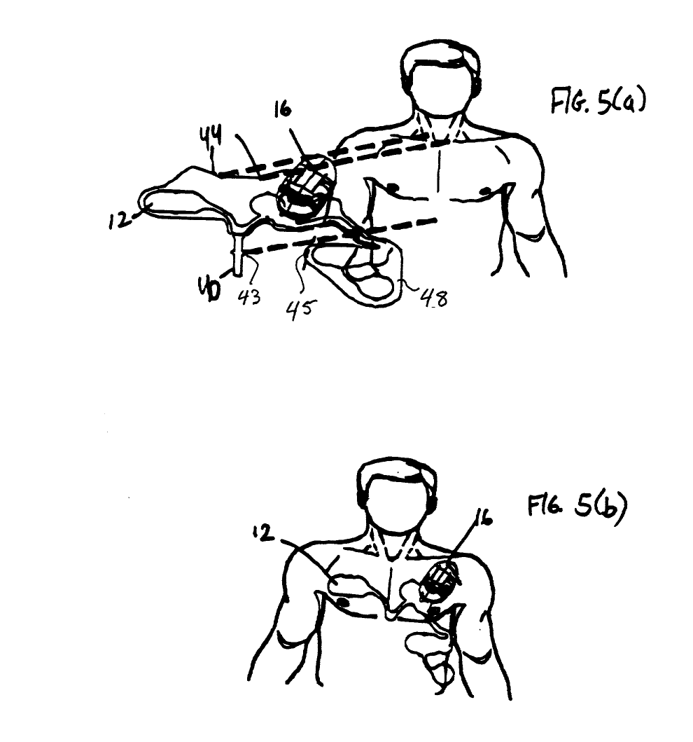

The sensor band 10 shown in FIGS. 2(a) and 2(b) comprises a 3 layer construction including a breathable membrane 32, screen printed polyester 34, and hydrogel disks 36 placed over electrodes 18. The polyester 34 has the tracks 20 and electrodes 18 screen printed onto its surface, and the tracks 20 are covered with a screen printable and a skin-friendly dielectric material that prevents moisture from affecting the electrical properties of the circuitry and prevents the polyester from causing any irritation to the patient. Circular hydrogel disks 36 are placed on each electrode 18 to provide electrical continuity between the patient's skin and the electrode 18. As explained below with respect to FIGS. 4(a)-4(h), both the polyester 34 and the hydrogel disks 36 are pre-cut into their shapes before the final construction stage. During the final construction stage, the polyester 34 and hydrogel disks 36 are sandwiched between the breathable membrane 32 and the topside release liner 38 shown in FIG. 3. The adhesive surface of the membrane 32 is used to attach the electrodes 18 to the patient's skin and to hold the polyester 34 in position. The membrane 32 is cut to shape, leaving the release liner on the skin side the same shape as the sensor 18. To help during the construction process, the polyester 34 has tabs and two or more alignment holes to help position the polyester 34 on the release liner on the skin side and to locate the final cut. On the other hand, the edge of the polyester sheet 34 may be used for alignment. The construction of the laminates of the sensor band 10 will be described in more detail below with respect to FIGS. 4(a)-4(h).

FIG. 3 illustrates a preferred embodiment of a release liner 38 on a side way from the patient's skin included for guiding the application of the sensor band 10 to the patient's chest. As shown, the release liner 38 is generally shaped the same as the sensor band 10 except that it is larger and includes a mid chest pointer 40 for providing horizontal alignment on the patient's chest (sternum) and a shaped neck section 42 including notches 44 for aligning with the patient's collarbones to provide vertical alignment on the patient's chest. Cut out portion 46 accepts the epatch 16 and the inter-connect 14. Preferably, the release liner 38 is semi-rigid, non-transparent, and holds the sensor band 10 in place during application and also provides support for interconnect 14, which may include a lower clip for accepting the epatch 16 as explained in detail in copending U.S. patent application Ser. No. 09/659,303, filed Sep. 12, 2001 and entitled “Disposable Vital Signs Monitoring Sensor Band with Reusable Electronics Module”. The aforementioned alignment points 40, 42, and 44 provide reference points for easy alignment of the sensor band 10 during application by the patient.

As also shown in FIG. 3, section 48 of the release liner 38 is further designed to tuck under the patient's left arm. Section 48 has a marker 45, which may be, for example, a notch or an arrow, that is used to position under arm section 48 relative to the patient's body. Specifically, marker 45 is designed to be aligned with a marker or bobble 43 located on chest pointer 40. Aligning marker 45 with bobble 43 serves to position under arm section 48 and the corresponding portion of sensor band 10 with the appropriate portion of the patient's body.

In an optional embodiment, a neck loop (not shown) may be applied to the release liner 38 so that the sensor band 10 and the release liner 38 are supported by the patient's neck during application. The neck loop also offers the option of providing further vertical adjustment on a per patient basis.

In addition, the sensor band 10 preferably includes alignment holes (not shown) at each electrode cluster to allow the option of aligning the sensor band 10 against body markings. Such alignment holes may also be used to mount the sensor band 10 on the release liner 38 during manufacture.

FIGS. 4(a)-4(h) illustrate the detailed method of constructing the disposable sensor band 10 in accordance with the invention. In FIGS. 4(a)-4(h), the figures are ordered so that the materials against the skin are applied last (FIG. 4(h)) while the materials further away from the skin are shown first. Of course, the outermost layer is the release liner 38 of FIG. 3 that is removed upon application to leave the sensor band 10. Thus, the membrane 32 shown in FIG. 4(a) is the outermost layer remaining upon removal of the release liner 38 of FIG. 3, while the hydrogel disks 36 of FIG. 4(h) are against the skin. It will be appreciated that the release liner 38 of FIG. 3 covers the whole area of the sensor band 10 and is cut out at the final cut along with the membrane 32.

FIG. 4(a) illustrates the outer shape of the membrane 32 after it has been cut. The yoke 51 connecting the chest electrodes to the underarm electrodes and the “V” 53 connecting the chest electrodes cut through membrane 32 and polyester 34. The electrode edge areas are just cut through the membrane 32, and the membrane 32 on the tail portion 50 where the interconnect 14 and epatch 16 attach is removed afterwards. As shown in FIG. 4(b), once the polyester 34 has had three layers of printing done and the membrane 32 has been added, the area where the membrane 32 adheres to the skin is cut out. This maintains the rigidity of the polyester 34 during manufacture and makes the final cut possible. Then, as shown in FIG. 4(c), silver ink 52 is screened onto the polyester 34 to the shape of the electrodes 18 and the tracks 20, including the tail area 50 where the interconnect 14 and epatch 16 attach. Dielectric ink 54 is next screened onto the polyester 34 to cover the silver tracks 20 but to leave the silver/silver chloride electrodes 18 exposed as shown in FIG. 4(d). The silver/silver chloride ink 56 is then screened onto the silver ink 52 as shown in FIG. 4(e), but only where the electrodes 18 are and over some of the polyester 34 to help hold it on, and not on the tracks 20. As shown in FIG. 4(f), an insulating layer 58 covers the screened dielectric 54 but has holes 60 cut out of it to allow the silver/silver chloride ink 56 to contact the hydrogel disks 36. The hydrogel disks 36, typically approximately 18 mm in diameter, are positioned on the silver/silver chloride ink 56 of each electrode 18 as shown in FIG. 4(g). The hydrogel 36 overlaps the silver/silver chloride ink 56, and the outer ring of hydrogel disks 36 adhere to the insulating material 58. Finally, FIG. 4(h) shows all layers together except for the release liners including release liner 38, which is shown in FIG. 3 and preferably is attached to the assembly of FIG. 4(h) so that it remains rigid during application but may be readily removed without dislodging the sensor band 10 once the sensor band 10 has been properly applied to the patient's skin.

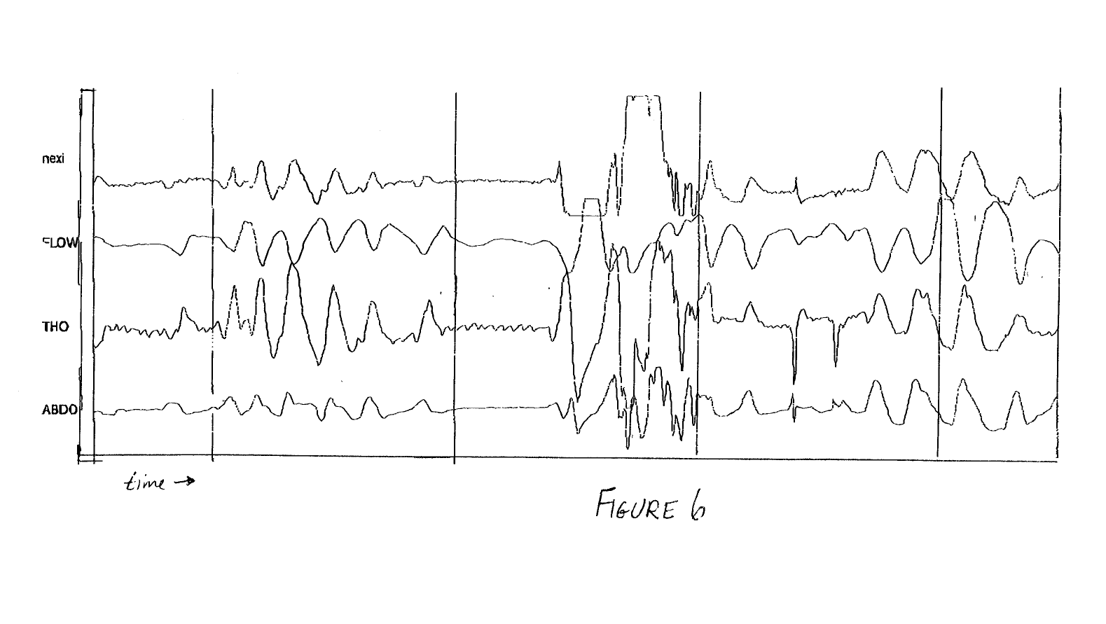

FIGS. 5(a) and 5(b) illustrate the technique for applying the disposable sensor band 10 of the invention by aligning the release liner 38 with the patient's collarbones and chest and then removing the release liner 38. Prior to application of the sensor band 10 to the patient's chest, the skin is prepared. Generally, the sensor band 10 is not applied to broken, damaged, or irritated skin and is not applied where lotions, oils, creams, perfumes, deodorants or powders have been used as they will adversely affect the adherence of the sensor band 10 to the patient's skin. If the patient's skin is hairy, then the skin site should be shaved and the skin site should be cleaned with an alcohol wipe and rubbed dry. Once the skin site is ready, a sensor band 10 of the proper size and with a charged battery (i.e., the sensor band 10 is still within its useful battery life) is selected for application and laid on a flat surface. A clip (interconnect) 14 for the epatch is then clipped to the tail portion 50 of the sensor band 10, and a VELCRO patch is placed (fabric side down) on the back of the clip 14 so that it covers the entire back of the epatch 16 including the clip 14. Preferably, the sensor band 10 is then checked to make certain that it is operational. Now the patient is ready to apply the sensor band 10 to his or her chest.

As shown in FIG. 5(a), the patient may use a mirror to check the position of the sensor band 10 so that the notches 44 in the release liner 38 line up with the collarbones at the base of the neck and that the mid chest pointer 40 points down the patient's sternum. Thereafter, marker 45 on underarm portion 48 of release liner 38 is aligned with bobble 43 on chest pointer 40. The patient then peels off the release liners over the hydrogel disks 36 and adhesive membrane 32 and the VELCRO backing strip. Starting with the chest and moving to the underarm area, the patient firmly presses and smoothes the release liner 38 and the epatch 16 into position on the skin. Once the sensor band 10 is attached to the patient's skin, the patient then gently peels and pulls away the release liner 38 from the sensor band 10 taking care that the sensors 18 and the epatch 16 remain firmly fixed to the patient's skin as shown in FIG. 5(b).

To remove the sensor band 10, the patient peels the epatch 16 away from the VELCRO patch on the body and gently peels and removes the sensor band 10 and epatch clip 14. The epatch 16 is removed from the sensor band 10 by pressing side clips and separating the two halves (the epatch 16 and clip interconnect 14). The epatch 16 is retained for future use with another sensor band 10. Finally, the VELCRO patch is removed and any residue is cleaned from the patient's body with soap and water. If further monitoring is to continue, then another sensor band 10 is applied to the patient as explained above.

The alignment technique of the invention leads easy, accurate alignment of the electrodes 18 in the proper pre-cordial positions. The improved accuracy of placement of the electrodes 18 is exemplified by the improved respiratory signals obtained using the on-body sensor 12 and diagonal impedance techniques in accordance with another aspect of the invention. In particular, the present inventors have observed that when on-body sensor 12 is placed using the techniques of the invention and an impedance is measured from the sensors 18 on the right breast diagonally to the sensors 18 under the patient's left axilla, that the resulting impedance measurement correlates quite nicely with air flow in and out of the patient's chest and hence is indicative of breathing. This so-called “diagonal impedance” in effect provides a combination of the chest and abdominal breathing movements by virtue of the locations of the sensors. Previously, the chest and abdominal breathing were measured separately by two sets of sensors: one across the chest cavity for measuring chest activity and one across the abdominal cavity for measuring diaphragm activity. The outputs of the two sets of sensors could be compared to discriminate the chest/respiratory effort from actual movement of air in and out of the patient's chest. The diagonal impedance respiration measurement technique in accordance with the invention allows combinatorial measurements to be made with one set of sensors and with unexpectedly improved accuracy.

The accuracy of the correlation of the diagonal impedance signal measured between the sensors 18 on the right breast and the sensors 18 under the left axilla to the air flow into the patient's lungs is illustrated in FIG. 6. As shown, the conventional abdominal impedance measurement (ABDO) and conventional thoracic impedance measurement (THO) vary independently with the air flow (FLOW), while the diagonal impedance measurement (NEXI) in accordance with the invention combines the effects of the ABDO and THO signals to correlate better to variations in the air flow. As shown, when the airflow volume dips or rises, the diagonal impedance signal correspondingly dips and rises. Accordingly, the diagonal impedance signal of the invention may be indicative of the patient's breathing, thus enabling the on-body sensor 12 of the invention to be useful in measuring other patient characteristics such as the presence of sleep apnea or other apnea events. Of course, the sensors 18 may be reversed such that one sensor 18 is on the left breast while the other sensor 18 is under the right axilla of the patient.

Although an exemplary embodiment of the invention has been described in detail above, those skilled in the art will readily appreciate that many additional modifications are possible in the exemplary embodiment without materially departing from the novel teachings and advantages of the invention. For example, the release liner 38 would be adapted to different anatomical features for application of the sensor band 10 to other parts of the body. All such modifications are intended to be included within the scope of this invention as defined in the following claims.

What is claimed is:

1. A method for positioning relative to a patient's chest a band containing at least one physiological sensor and having a release liner attached thereto, comprising the following steps:
aligning a tab portion of the release liner with the patient's sternum;
aligning a first marker on an underarm section of the release liner with a second marker on the tab portion of the release liner;
attaching the band to the patient's skin; and
removing the release liner.

2. The method of claim 1, further comprising the step of aligning markings on a neck shaped portion of the release liner with the patient's collarbones.

3. The method of claim 1, further comprising the step of placing a neck loop of said release liner around the patient's neck for aligning said release liner on the patient's body.

4. A method of measuring a patient's breathing using a sensor band placed on a patient's chest, said sensor band containing at least a first physiological sensor adapted for placement on one of the patient's breasts and a second physiological sensor adapted for placement under the patient's axilla on a side of the patient opposite said one breast, comprising the following steps:
attaching the sensor band to the patient's chest so that said first physiological sensor is placed over said one breast and said second physiological sensor is placed under the patient's axilla on the side of the patient opposite said one breast; and
measuring impedance between the first and second physiological sensors.

5. A method as in claim 4, wherein said sensor band comprises a release liner and said attaching step comprises the steps of:
aligning a tab portion of the release liner with the patient's sternum;
aligning a first marker on an underarm section of the release liner with a second marker on the tab portion of the release liner;
attaching the band to the patient's skin; and
removing the release liner.

6. A sensor band for application to a patient's chest, comprising:
at least one sensor to be applied to a predetermined position on the patient's chest;
electronics circuitry that processes physiological parameter signals from said at least one sensor for at least one of storage and transmission to a monitoring location; and
a release liner having alignment points that are aligned with physiological features of the patient during application to the patient's skin to provide for horizontal and vertical alignment of the sensor band to the patient's skin, wherein said release liner comprises a pointer tab that aligns with the patient's sternum and has a marker thereon, and an underarm section having a second marker located thereon, whereby said second marker may be aligned with said first marker to position said underarm section at the proper position on the patient's body.

7. A sensor band as in claim 6, wherein said release liner further comprises a shaped neck section with notches that align with the patient's collarbones.

8. A sensor band as in claim 6, wherein said release liner further comprises a neck loop that fits around the patient's neck during application.

9. A sensor band as in claim 6, further comprising a breathable membrane that has holes that align said membrane to said release liner.

10. A sensor band as in claim 6, further comprising a polyester layer, said polyester layer providing rigidity to said sensor band.

11. A sensor band as in claim 6, wherein said electronics circuitry is mounted in an assembly separable from said sensor band and connected to said at least one sensor by a connector and at least one circuit track.

12. A sensor band as in claim 9, wherein said breathable membrane has holes that are aligned with body markings on the patient's skin.

13. A sensor band as in claim 6, wherein said at least one sensor comprises a first physiological sensor adapted for placement on one of the patient's breasts and a second physiological sensor adapted for placement under the patient's axilla on a side of the patient opposite said one breast, and wherein said electronics circuitry processes impedance signals measured between said first and second physiological sensors.

14. A sensor band for application to a patient's chest to measure the patient's breathing, comprising:
a first physiological sensor adapted for placement on one of the patient's breasts and a second physiological sensor adapted for placement under the patient's axilla on a side of the patient opposite said one breast; and
electronics circuitry that processes impedance signals measured between said first and second physiological sensors for at least one of storage and transmission to a monitoring location.

15. A sensor band as in claim 14, further comprising a release liner having alignment points that are aligned with physiological features of the patient during application to the patient's skin to provide for horizontal and vertical alignment of the sensor band to the patient's skin, wherein said release liner comprises a pointer tab that aligns with the patient's sternum and has a marker thereon, and an underarm section having a second marker located thereon, whereby said second marker may be aligned with said first marker to position said underarm section at the proper position on the patient's body for breathing measurement.
